(19) United States
(12) Patent Application Publication
(10) Pub. No.: US 2026/0036515 A1
(43) Pub. Date: Feb. 5, 2026

(54) METHOD FOR OBTAINING A MODEL FOR A SPECTROMETER OR A SPECTROSCOPE AND METHOD FOR EVALUATING A SPECTRUM OF A GAS OR GAS MIXTURE

(71) Applicant: Endress+Hauser Optical Analysis, Inc., Ann Arbor, MI (US)

(72) Inventor: Kevin Ludlum, Freiburg (DE)

(21) Appl. No.: 19/101,418

(22) PCT Filed: Aug. 18, 2023
(86) PCT No.: PCT/US2023/072455

§ 371 Date: Feb. 5, 2025

(30) Foreign Application Priority Data
Aug. 19, 2022 (DE) 102022121066.9

(51) Int. Cl. G01N 21/3504; H04L 9/40

(52) U.S. Cl. CPC G01N 21/3504; H04L 63/0428; G01N 2201/129

(57) ABSTRACT

A method for obtaining a model for a spectrometer or spectroscope, wherein a model comprises a relationship between a spectrum of a gas mixture or gas and parameters of the gas mixture or gas includes acquiring a reference spectrum of a predetermined gas or gas mixture using the spectrometer or spectroscope and parameters of the predetermined gas or gas mixture, encrypting the spectrum and/or the parameters using a homomorphic encryption method, and analyzing the spectrum and parameters without decoding the spectrum, or parameters. The method also includes performing one or more of the following steps with respect to a model based on the analysis, wherein a model contains a relationship between the spectrum and the parameters of the gas mixture: selection of a model from a large number of models created in advance, adjusting a previously created model, and recreating a model.

### CROSS-REFERENCE TO RELATED APPLICATION

The present application is related to and claims the benefit priority of German Patent Application No. 102022121066.9, filed Aug. 19, 2022, the entire contents of which are incorporated herein by reference.

### TECHNICAL FIELD

The present disclosure relates to a method for obtaining a model for a spectrometer or a spectroscope, wherein a model contains a relationship between a spectrum of a gas mixture or gas and parameters of the gas mixture or gas, in particular the composition and/or concentrations of the individual components of the gas mixture. The present disclosure further relates to a method for evaluating a spectrum of a gas or gas mixture detected by a spectrometer or spectroscope by means of the model obtained by the aforementioned method.

### BACKGROUND

Induced radiation effects such as Raman scattering and fluorescence have become extremely valuable tools for the nondestructive determination of molecular constituents. Raman spectroscopy is an established and practical method of chemical analysis and characterization that can be applied to many different chemical fabrics. As a real-time, non-destructive technique, Raman spectroscopy is compatible with a wide range of samples, including opaque solids, aqueous solutions, emulsions and gases, without the need for sample preparation.

In addition to Raman spectrometers, a variety of other types of spectrometers or spectroscopes exist, including IR spectroscopes and NIR spectroscopes.

Each type of spectrometer or spectroscope behaves uniquely in recording a spectrum of a mixture of gases. A model is used to evaluate the spectrum, in particular to obtain parameters of a gas mixture such as the composition and/or the concentrations of the individual components of the gas mixture. Such a model contains one or more regression algorithms or parameters for one or more such regression algorithms. The model has parameters for a regression algorithm, wherein the regression algorithm calculates the parameters from the spectrum.

Creating a model for a customer currently requires a lot of detailed, hands-on work, often using sensitive customer data. The content of this data indicates which chemical constituents are present and in what quantities, particularly for an experienced chemometrician. A company may therefore be reluctant to disclose information that could reveal trade secrets. This makes it difficult to offer modeling as a service. Since this service can be valuable to a company and help expand the customer base for spectroscopic apparatus, there is a need to be able to create models without compromising privacy for the customer. A variety of encryption methods exist to hide the content of information, but none of these methods are designed to address the unique privacy needs of spectroscopic applications.

When dealing with encrypted customer data for spectroscopic applications, particularly for Raman applications, there are two main points to consider:

The model creation process requires many hands-on steps that are typically performed by a chemometrician or expert modeler.

In addition, some additional information must be hidden from the model builder to ensure that customer data is protected (particularly which wavelengths or frequencies of the spectrum are relevant to the model).

### SUMMARY

Based on this problem, the object of the present disclosure is to present a method that allows a model builder to create a model for a spectrometer, or spectroscope, of a customer without requiring knowledge of the customer's data.

The object is accomplished by a method for obtaining a model for a spectrometer or a spectroscope, wherein a model comprises a relationship between a spectrum of a gas mixture or gas and parameters of the gas mixture or gas, in particular the composition and/or concentrations of the individual components of the gas mixture:
acquiring a reference spectrum of a predetermined gas or gas mixture by means of the spectrometer or spectroscope and parameters of the predetermined gas or gas mixture, in particular the composition and/or concentrations of concentrations of the individual components of the predetermined gas mixture;
encrypting the spectrum and/or the parameters by means of a homomorphic encryption method;
analyzing the spectrum and parameters without decoding the spectrum, or parameters;
performing one or more of the following steps with respect to a model based on the analysis, wherein a model contains a relationship between the spectrum and the parameters of the gas mixture:
   i. selection of a model from a large number of models created in advance,
   ii. adjusting a previously created model, and
   iii. recreating a model.

Thus, according to the present disclosure, a homomorphic encryption technique is used to hide the customer reference spectrum from the model builder. However, due to the properties of the reference spectrum encoded by means of the homomorphic method, it is still possible for the modeler to analyze the spectrum and create customized models-without the modeler being able to see the reference spectrum along with the frequency content associated in the reference spectrum (and in some cases, not even the parameters of the model itself).

Spectrometers and spectroscopes are measuring instruments which record a spectrum of a gas mixture and enable the individual components of a gas mixture to be identified with high accuracy. While a spectroscope provides information about which components are present in a gas mixture, spectrometers also provide a statement about the quantity of the respective components—in other words, they indicate for their measurement range how large the radiation intensity is at the respective observed wavelength.

In accordance with an advantageous further development of the method according to the present disclosure, it is provided that the steps of recording and encrypting are performed by a first instance, in particular a customer, wherein the encrypted spectrum and/or the encrypted parameters are transmitted to a second instance, in particular a service provider, wherein the steps of analyzing and performing the steps regarding the model are performed by the second instance. One or more computers are provided for this purpose, with which the respective method steps are performed. It may also be provided to access a cloud from the instances IN1, IN2 on which one or more of the method steps are performed.

In accordance with an advantageous embodiment of the method according to the present disclosure, it is provided that the reference spectrum is shuffled by the first instance prior to encryption, wherein information about the type of shuffling is collected, wherein the information is not transmitted to the second instance. For example, the reference spectrum is divided into frequency intervals, which are subsequently interchanged with respect to the order. This increases security because even if the reference spectrum is decoded, the exact reference spectrum cannot be obtained.

In accordance with an advantageous embodiment of the method according to the present disclosure, it is provided that a key pair comprising a private key and a public key is created on the first instance, wherein the public key is transmitted to the second instance, wherein the model is encrypted by the second instance by means of the public key and is decryptable by the private key. This increases security by preventing a third party from interpreting the model, for example in the event of a hack.

In accordance with an advantageous embodiment of the method according to the present disclosure, it is provided that the model has parameters for a regression algorithm, wherein the regression algorithm calculates the parameters from the spectrum.

In accordance with an advantageous embodiment of the method according to the present disclosure, it is provided that a machine learning or AI algorithm is used for the steps of selecting, adapting or recreating the model.

Furthermore, the object is solved by a method for evaluating a spectrum of a gas or gas mixture detected by a spectrometer or spectroscope by means of the model obtained by the aforementioned method according to the present disclosure, wherein parameters of the gas or gas mixture are obtained by evaluating the spectrum.

In accordance with an advantageous embodiment of the method according to the present disclosure, it is provided that the model is transmitted to the first instance and wherein the evaluation is performed by the first instance.

### BRIEF DESCRIPTION OF THE DRAWINGS

The present disclosure is explained in greater detail with reference to the following figures. in which:

FIG. 1 shows a flowchart of a first embodiment of the method according to the present disclosure; and

FIG. 2 shows a flowchart of a second embodiment of the method according to the present disclosure.

### DETAILED DESCRIPTION

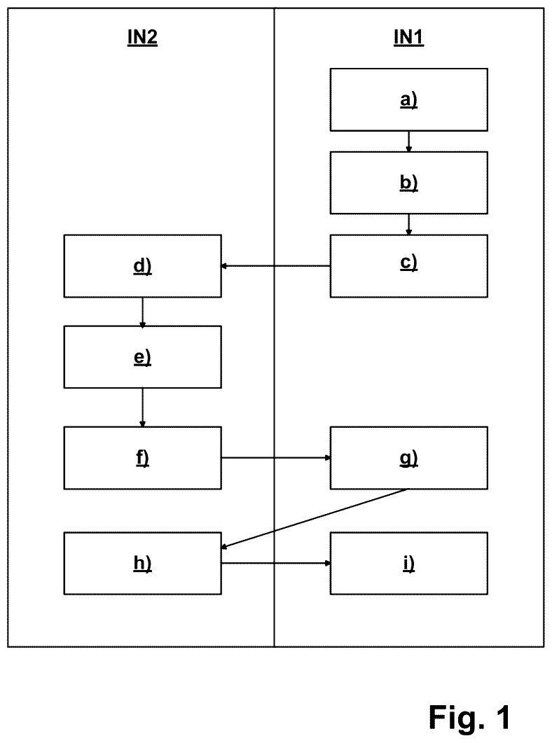

In FIG. 1, in a first embodiment of the method according to the present disclosure, a suitable model is selected and adapted for a spectrometer, or a spectrometer. A model is specific for a spectrometer or a spectroscope and describes a relationship between a spectrum detected by the spectrometer or the spectroscope and parameters of a gas mixture, in particular the composition and/or concentrations of the individual components of the gas mixture whose spectrum was detected by means of the spectrometer. In other words, the model makes it possible to determine the parameters of a gas mixture from its detected spectrum.

The spectrometer, or spectroscope, is used by the customer, referred to here as the first instance IN1.

The task of selecting and fitting the appropriate model is given to a model builder, referred to here as the second instance IN2.

In a first method step a) the customer acquires a reference spectrum of a known gas mixture containing at least 2 components by means of the spectrometer, or spectroscope. A known gas mixture means that the customer knows exactly the parameters of the gas mixture, for example the composition and/or concentrations of the individual components of the gas mixture. A spectrum usually has a frequency or wavelength on one axis, while the other axis represents an intensity. The intensity is measured by the spectrometer or spectroscope over a frequency or wavelength range. Different gas mixtures have different characteristic curves. By means of the model, a statement can be made about how the spectrometer, or spectroscope, captures and extends these characteristic progressions-since each spectrometer, or spectroscope, represents these progressions differently. By applying the model to spectra of unknown gas mixtures, their parameters can thus also be determined. Thus, in particular, the model contains parameters for a regression algorithm, which regression algorithm can calculate the parameters from the spectrum.

In method step b), a key pair is created in the first instance IN1. This consists of a public key and a private key. By means of the public key, information or data can be encrypted. The public key is used to decrypt the encrypted data.

Since the reference spectrum and, if applicable, the parameters are sensitive information that should not be released to the model builder, the reference spectrum and, if applicable, the parameters are encrypted in method step c) by means of a homomorphic encryption method. The homomorph-encrypted data can be processed, or offset, by its special property without the processing second instance IN2 needing knowledge of the unencrypted file contents. The result data that has been processed or charged can be decoded again by the customer, whereby the processing or charging remains in effect.

Partially homomorphic encryption methods or fully homomorphic encryption methods can be considered for this purpose, for example. Homomorphic encryption methods, or cryptosystems, can be classified by their homomorphism properties:

Partially homomorphic encryption methods exist, for example, as additively homomorphic encryption methods (partial) with the following property:

$$m ( a ) ⊕ m ( b ) = m ( a + b ) ;$$

or as multiplicatively homomorphic encryption methods (partial) having the following property.

$$m ( a ) ⊗ m ( b ) = m ( a \times b ) .$$

In addition, fully-homomorphic encryption methods exist that have both additive and multiplicative homomorphic properties.

The reference spectrum encrypted in this manner and the public key, as well as, if applicable, the parameters of the known gas mixture, are transmitted to the second instance IN2 and received by it in method step d).

Subsequently, the encoded reference spectrum is analyzed in method step e) and, if necessary, correlated with the parameters. Based on this analysis, a suitable model for the spectrometer, or spectroscope, is selected from a variety of models. This is still adjusted in method step f) if necessary.

For method steps e) and f), one or more AI or machine learning algorithms are used, which have been learned in advance on a variety of spectra and, if necessary, parameters. Due to the homomorphic encryption method used, the second instance IN2 does not have to resolve the encryption in order to perform method steps e) and f). Subsequently, the instance IN2 generates results of these method steps, particularly information regarding the model (e.g. information regarding the regression), encrypts them by means of the public key and transmits them to the first instance IN1.

After receipt of the results by the first instance, they are decoded in method step g). Subsequently, their plausibility is checked and in particular the model performance is tested. If the results are not plausible for the customer, a corresponding feedback is reported to the second instance IN2, thus repeating method steps e) and f). In case the customer confirms the plausibility, a corresponding feedback is reported to the second instance IN2.

In the subsequent method step h), the selected, or adapted, model is encrypted by means of the public key and transmitted to the first instance IN1. Encryption means that the sensitive information contained in the model cannot be interpreted by unauthorized persons.

In the final method step i), the model is decrypted by the first instance IN1 by means of the private key. Subsequently, a final assessment of the model is made. For this purpose, for example, additional spectra of further known gas mixtures are recorded. By means of the model, the parameters of the respective gas mixtures are subsequently determined and further known gas mixtures are selected using the actually known parameters of the further gas mixtures. If the final assessment is successful, the spectrometer, or spectroscope, is put into operation with this model.

In case the final evaluation is not successful, the method can be repeated from method step e).

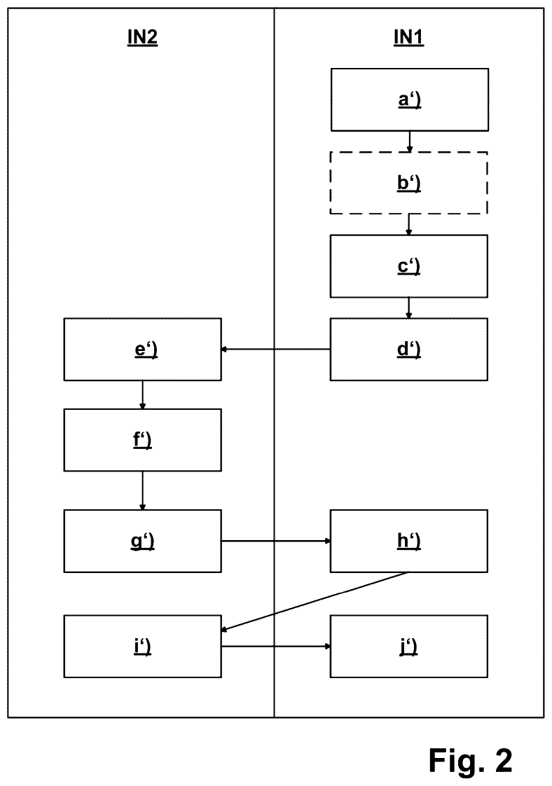

In FIG. 2, in a second embodiment of the method according to the present disclosure, a suitable model is created for the spectrometer, or spectroscope, and adapted if necessary. The method is substantially similar to the method described in FIG. 1, but differs in various method steps.

In a first method step a′) the customer acquires the reference spectrum of the known gas mixture, which contains at least 2 components, by means of the spectrometer, or the spectroscope.

In the optional method step b′), the reference spectrum is mixed in the first instance IN1. This means, for example, that the frequencies are no longer arranged in ascending order, but are divided into frequency intervals, which are interchanged with each other in terms of sequence. Information on how exactly the mixing was done is stored by the first instance IN1. With the help of this information, the reference spectrum can be sorted back into the correct order.

In method step c′), the first instance IN1 creates the key pair consisting of the public key and the private key.

Subsequently, the reference spectrum and, if necessary, the parameters in method step d′) are encrypted by means of the homomorphic encryption method.

The encrypted reference spectrum is then received in method step e′) together with the public key and analyzed in method step f) and, if necessary, correlated with the parameters. On the basis of this analysis, one or more suitable models for the spectrometer or spectroscope are created and, if necessary, adapted in method step f).

For method steps f) and g′), one or more AI or machine learning algorithms are used, which have been learned in advance on a variety of spectra and, if necessary, parameters. Due to the homomorphic encryption method used, the second instance IN2 does not have to resolve the encryption in order to perform the method steps f) and g′). The AI or machine learning algorithm(s) are also capable of processing the mixed and encrypted reference spectrum accordingly. Subsequently, the instance IN2 generates results of these method steps, particularly information regarding the model (e.g. information regarding the regression), encrypts them by means of the public key and transmits them to the first instance IN1.

After receipt of the results by the first instance, they are decoded in method step g). Subsequently, it is checked which of the models has the highest model performance. For this purpose, the results are compared with ferences. If the results are not satisfactory for the customer, a corresponding feedback is reported to the second instance IN2, so that the method steps f) and g′) are repeated. The finally selected model is communicated to the second instance IN2.

In the subsequent method step i′), the selected model is encrypted by means of the public key and transmitted to the first instance IN1. Encryption means that the sensitive information contained in the model cannot be interpreted by unauthorized persons.

In the final method step j′), the model is decrypted by the first instance IN1 by means of the private key. Subsequently, a final assessment of the model is made. For this purpose, for example, additional spectra of further known gas mixtures are recorded. By means of the model, the parameters of the respective gas mixtures are subsequently determined and further known gas mixtures are selected using the actually known parameters of the further gas mixtures. If the final assessment is successful, the spectrometer, or spectroscope, is put into operation with this model.

In case the final evaluation is not successful, the method can be repeated from method step f*).

The method according to the present disclosure is not limited to the two embodiments mentioned. For the person skilled in the art, it goes without saying that he can also combine various of the method steps mentioned in only one of the embodiments in each case.

On the side or in the instances IN1, IN2 means that there are one or more computers with which the method steps are performed. This also includes that the instances IN1, IN2 can access a cloud on which one or more of the method steps can be performed.

1-8. (canceled)

9. A method for obtaining a model for a spectrometer or a spectroscope, wherein the model comprises a relationship between a spectrum of a gas mixture or gas and parameters of the gas mixture or gas, comprising:
acquiring a reference spectrum of a predetermined gas or gas mixture using the spectrometer or spectroscope and parameters of the predetermined gas or gas mixture;
encrypting the spectrum and/or the parameters using a homomorphic encryption method;
analyzing the spectrum and parameters without decoding the spectrum, or parameters;
performing one or more of the following steps with respect to a model based on the analysis, wherein a model contains a relationship between the spectrum and the parameters of the gas mixture:
selection of a model from a large number of models created in advance,
adjusting a previously created model, and
recreating a model.

10. The method according to claim 9, wherein the steps of recording and encrypting are performed by a first instance, wherein the encrypted spectrum and/or parameters are transmitted to a second instance, wherein the steps of analyzing and performing the steps regarding the model are performed by the second instance.

11. The method according to claim 10, wherein the reference spectrum is intermixed by the first instance prior to scrambling, wherein information about the type of intermixing is collected, wherein the information is not transmitted to the second instance.

12. The method according to claim 10, wherein a key pair consisting of a private key and a public key is created on the first instance, wherein the public key is transmitted to the second instance, wherein the model is encrypted by the second instance using the public key and is decryptable by the private key.

13. The method according to claim 9, wherein the model has parameters for a regression algorithm, wherein the regression algorithm calculates the parameters from the spectrum.

14. The method according to claim 9, wherein a machine learning or AI algorithm is used for the steps of selecting, adjusting, or recreating the model.

15. The method of evaluating a spectrum of a gas or gas mixture detected by a spectrometer or spectroscope using the model obtained by a method according to claim 9, wherein parameters of the gas or gas mixture are obtained by evaluating the spectrum.

16. The method according to claim 15, wherein the model is transmitted to the first instance and wherein the evaluation is performed by the first instance.
